(19) United States
(12) Patent Application Publication
(10) Pub. No.: US 2012/0199256 A1
(43) Pub. Date: Aug. 9, 2012

(54) SUSPENSIONS OF HEXANITROHEXAAZAISOWURTZITANE CRYSTALS, PRODUCTION OF SAID SUSPENSIONS AND PRODUCTION OF PYROTECHNIC OBJECTS

(75) Inventors: Florent Muscatelli, Vert Le Petit (FR); Philippe Lescop, Vert Le Petit (FR)

(73) Assignees: EURENCO, Paris (FR); SME, Paris (FR)

(21) Appl. No.: 13/497,170

(22) PCT Filed: Sep. 28, 2010
(86) PCT No.: PCT/FR2010/052027

§ 371 Date: Apr. 26, 2012

(30) Foreign Application Priority Data
Sep. 29, 2009 (FR) 0956736

(51) Int. Cl. C07D 487/22; C06B 25/34

(52) U.S. Cl. 149/92; 540/554

(57) ABSTRACT

The present invention relates to suspensions of hexanitrohexaazaisowurtzitane crystals in a liquid phase (said liquid phase is composed, to at least 85% by weight, of a nonsolvent for hexanitrohexaazaisowurtzitane, said nonsolvent consisting of at least one nonflammable hydrofluoroether, and includes from 0 to less than 15% by weight of an organic solvent for hexanitrohexaazaisowurtzitane, more volatile than said nonsolvent, chosen from the group consisting of esters, nitriles, ketones and their mixtures), the production of said suspensions and their use to manufacture pyrotechnic objects. The use of said at least one hydrofluoroether as nonsolvent is particularly appropriate.

The present invention relates to:

suspensions of hexanitrohexaazaisowurtzitane crystals in a liquid phase (including a novel nonsolvent for said hexanitrohexaazaisowurtzitane),

a process for producing said suspensions, and

the manufacture of pyrotechnic objects using such suspensions.

The invention comes within the field of powders, propellants and explosives used in particular in the armaments industry.

There have existed, for some years, numerous publications relating to 2,4,6,8,10,12-hexanitro-2,4,6,8,10,12-hexaazatetracyclo-(5.5.0.0^{5,9}.0^{3,11})dodecane, also known as hexanitrohexaazaisowurtzitane or CL20. These publications describe the various polymorphic forms of this compound (this is because it is known that hexanitrohexaazaisowurtzitane can be obtained in four crystalline polymorphic forms: beta, alpha, gamma and epsilon), the physical, chemical and detonating properties of this compound and the use of said compound in explosive compositions, propellants or powders for weapons.

It is the epsilon polymorphic form of said compound (CL20ε) which has the highest density (2.04 g/cm³) and which is thus of the greatest interest, in particular for use in pyrotechnic compositions.

Patent application EP 0 913 374 describes a process for producing CL20ε according to the following reaction stages:

first of all, a saturated solution of CL20 of any polymorphic form, preferably other than the epsilon form, is prepared in a mixture comprising, on the one hand, an organic solvent for CL20 chosen from the group consisting of esters, nitriles, ethers, ketones other than acetone and their mixtures and, on the other hand, a nonsolvent for CL20 chosen from the group consisting of aliphatic hydrocarbons, aromatic hydrocarbons and their mixtures, said solvent for CL20 being more volatile than said nonsolvent (i.e. exhibiting a lower saturated vapor pressure than that of the nonsolvent), and said solvent and said nonsolvent being miscible in the proportions used (for the preparation of said mixture, of said solution),

this saturated solution is subsequently seeded with a few crystals of CL20ε, then

the solution is concentrated by complete or partial evaporation of the solvent, which brings about the appearance of CL20ε crystals, which remain in suspension in the mixture enriched in nonsolvent.

These crystals can subsequently be recovered by any normal method, such as filtration.

Mention may be made, as examples of organic solvents for CL20 which can be used according to said process, of methyl formate, methyl acetate, ethyl acetate, isopropyl acetate, acetonitrile, ethyl acetate/acetonitrile mixtures, tetrahydrofuran (THF) and methyl ethyl ketone.

Mention may be made, as examples of nonsolvents for CL20, of toluene, xylenes, alkanes, such as hexane, heptane and octane, and halogenated aliphatic hydrocarbons, in particular chlorinated aliphatic hydrocarbons, such as 1,2-dichloroethane.

The solvent/nonsolvent pair: ethyl acetate/toluene, is particularly preferred.

The solvent/nonsolvent ratio by volume is generally between 10/90 and advantageously between 15/85 and 35/65.

During the concentration of the solution by evaporation of the organic solvent, the temperature advantageously does not exceed 50° C., this being with reference to the purity of the CL20ε crystals desired.

On conclusion of the implementation of the process according to EP 0 913 374, a suspension of CL20ε crystals in a nonsolvent or a solvent/nonsolvent mixture enriched in nonsolvent is thus obtained, said nonsolvent being chosen from aliphatic hydrocarbons, aromatic hydrocarbons and their mixtures, consisting in particular of at least one of the compounds listed above, more particularly of toluene. Such non-solvents are flammable.

Thus, although being satisfactory with regard to the industrial production of CL20, the process according to EP 0 913 374 exhibits the disadvantage of using a flammable nonsolvent. This complicates the implementation of said process and more particularly that of the phases subsequent to said process: phases of storage, transportation and use of the CL20 produced (said phases of storage and transportation being, in absolute terms, only optional). At the end of the process, the CL20 thus cannot be stored in suspension in the nonsolvents (or in the solvent/nonsolvent mixtures) selected in application EP 0 913 374. For this reason, at the end of the process according to EP 0 913 374, the CL20ε suspension is generally washed with water, so as to remove said nonsolvent (or said mixture). The CL20ε produced is subsequently generally phlegmatized with water (at a level of approximately 20%) for the transportation and storage thereof. The subsequent removal of the water, during the manufacture of pyrotechnic objects, is complex (it is generally carried out by drying in a bed) and the residual traces of water are capable of bringing about undesirable reactions with some ingredients of the formulation of said pyrotechnic objects, in particular with crosslinking agents of the binder, such as polyisocyanates.

In such a context, it has appeared opportune to the inventors to look for a nonsolvent for CL20 which is nonflammable, which is suitable for the implementation of the process according to application EP 0 913 374 for producing suspensions of CL20 crystals (of ε polymorphic or other form) and which is advantageously optimized with reference to carrying out the phase or phases subsequent to said process: the phase of use of said suspensions in the manufacture of pyrotechnic objects and, generally, the phase(s) prior to said use, to said manufacture (storage or(and) transportation phase(s)), mentioned above. The requirements of such a compound, optimized with reference to all the phases indicated, comprise the following specifications:

of being, very clearly, a nonsolvent for CL20, which is suitable for preparing a solution according to the teaching of application EP 0 913 374,

of being nonflammable,

of having a phlegmatizing action with regard to the charge of CL20 in suspension and thus of making possible the risk-free storage and transportation of said charge of CL20 in suspension, and

of being able to be used in a process for the manufacture of pyrotechnic objects (that is to say, easy to extract, during the manufacture of such pyrotechnic objects, and inert, if traces thereof remain in the pyrotechnic object manufactured), and ideally,

of being relatively nontoxic to the environment (in contrast to the toluene used to date).

With reference to such requirements, the present invention provides an improvement to the teaching of patent application EP 0 913 374.

According to its first subject matter, the present invention relates to novel suspensions of hexanitrohexaazaisowurtzitane crystals in a liquid phase. These suspensions are of the type of those obtained on conclusion of the implementation of the process according to EP 0 913 374. Characteristically, their liquid phase is composed, to at least 85% by weight, of a nonsolvent for hexanitrohexaazaisowurtzitane, said nonsolvent consisting of at least one nonflammable hydrofluoroether, and includes from 0 to less than 15% by weight of an organic solvent for hexanitrohexaazaisowurtzitane, which is more volatile than said nonsolvent (i.e. exhibiting a lower saturated vapour pressure than that of said nonsolvent), chosen from the group consisting of esters, nitriles, ketones and their mixtures. Said solvent (when such a solvent is present (in any case, at less than 15% by weight)) and said nonsolvent are miscible in the proportions in which they are involved in the suspensions of the invention. Said at least one nonflammable hydrofluoroether thus replaces the aliphatic hydrocarbons, aromatic hydrocarbons and their mixtures used as nonsolvent in the process according to EP 0 913 374.

The suspensions of the invention are thus capable of existing according to two alternative forms: their liquid phase comprises or does not comprise (virtually does not comprise) a solvent for hexanitrohexaazaisowurtzitane. According to a first alternative form, they include a significant amount (greater than or equal to 4% by weight) of solvent, but less than 15% by weight. Said solvent is advantageously chosen from ethyl acetate and acetone and very advantageously consists of ethyl acetate. According to another alternative form, the suspensions of the invention do not include or include very little (less than 4% by weight, reference may be made to traces) solvent. Said at least one nonflammable hydrofluoroether constitutes their liquid phase.

Hydrofluoroethers (HFEs) are chemical compounds composed of hydrogen, fluorine and carbon atoms (they comprise neither chlorine nor bromine nor iodine) with an ether structure. They are known to a person skilled in the art. In the context of the present invention, nonflammable hydrofluoroethers (HFEs) have been selected. Such nonflammable hydrofluoroethers (HFEs) are advantageously chosen from those whose use is recommended in dry-cleaning compositions according to application WO 00/36206, i.e. those described in application WO 96/22356 and those described in U.S. Pat. No. 6,658,962. HFEs are generally known to be nonflammable if their number of fluorine atoms divided by the sum of the number of hydrogen atoms and the number of carbon-carbon bonds is greater than or equal to 0.8. In this respect, reference may be made to the teaching of said application WO 00/36206. The suspensions of the present invention thus include, as nonsolvent, an HFE or a mixture of HFEs exhibiting this characteristic.

The preferred nonflammable HFEs of the invention have a boiling point of between 40° C. and 275° C., advantageously of greater than 100° C., in order to make possible the storage, transportation and use of the suspensions of the invention under temperature conditions similar to those of aqueous suspensions. Said nonflammable HFEs, like all HFEs:

exhibit a low saturated vapor pressure and are thus well suited to the production of the suspensions of the invention by a solvent/nonsolvent crystallization process including the evaporation of said solvent which is more volatile than said nonsolvent (see hereinbelow the second subject matter of the invention);

are solvents which are inert and which have a low latent heat of evaporation (much lower than that of water), which makes possible the direct use of the suspensions of the invention as starting materials in the manufacture of pyrotechnic solid compounds (see hereinbelow the third subject matter of the invention).

Among nonflammable HFEs, those of segregated type (nonflammable segregated hydrofluoroethers) are preferred. These compounds have been described in applications WO 96/22356 and WO 00/36206. Their structure comprises at least one perfluoroalkane compound, perfluorocycloalkane compound, perfluoroalkane compound including a perfluorocycloalkyl group, or perfluoroalkane compound including a perfluorocycloalkylene group, such compound being mono-, di- or trisubstituted by an alkoxy group.

Among nonflammable HFEs, nonflammable segregated HFEs, 2-trifluoromethyl-3-ethoxy-1,1,1,2,3,4,4,5,5,6,6,6-dodecafluorohexane (known more simply hereinbelow as 2-trifluoromethyl-3-ethoxydodecafluorohexane), with the semi-structural formula (CF₃)C₆F₁₂OC₂H₅, is more particularly preferred. It is referenced under the number CAS 297730-93-9. Its boiling point is 128° C. and its saturated vapor pressure is 6 mmHg at 20° C. Its thermodynamic properties allow it to be used in the context of the implementation of the process according to EP 0 913 374, in place of the (flammable) organic solvent indicated. This chemical compound is produced in particular by 3M and is sold by this company under the commercial name HFE 7500. The solubility of hexanitrohexaazaisowurtzitane in 2-trifluoromethyl-3-ethoxydodecafluorohexane is 0.012% by weight at 20° C.; this is thus clearly a nonsolvent for CL20.

Nonflammable (nonsolvent) HFEs are sufficiently miscible with esters, nitriles, ketones and their mixtures (solvents) for the production of a mixture (within the meaning of application EP 0 913 374), in order to obtain the desired result.

Furthermore, the inventors have demonstrated (see point C of the examples below) that nonflammable HFEs have a proven phlegmatizing effect on the charge of CL20 crystals for a content by weight of at least 35%, advantageously of at least 50%. The suspensions of the invention thus advantageously include at least 35% by weight of this novel nonsolvent (for at most 65% by weight of charge (of crystals) of CL20), very advantageously at least 50% by weight of this novel nonsolvent (for at most 50% by weight of charge (of crystals) of CL20).

Said nonflammable HFEs thus develop a phlegmatizing action. Said nonflammable HFEs thus advantageously replace the nonsolvents of the process according to EP 0 913 374 and the water, a nonsolvent normally used as phlegmatizing agent for the storage and transportation of CL20 (see above). The suspensions of the invention can thus, if necessary, be stored and transported without risk.

A person skilled in the art has already, without any doubt, grasped the great advantage of the suspensions of the invention.

It should be noted, incidentally, that the suspensions of the invention include crystals of CL20 of any polymorphic form, that they advantageously include crystals of CL20 ε.

According to its second subject matter, the present invention relates to a process for producing such suspensions. It is a process analogous to the process according to EP 0 913 374. It constitutes an improvement to said process. Characteristically, it is carried out with a novel nonsolvent, said at least one nonflammable HFE. The process, for producing a suspension of crystals of CL20 in a nonsolvent, of the invention comprises:

the preparation of a saturated solution of CL20 of any polymorphic form in a mixture comprising, on the one hand, an organic solvent for CL20 chosen from the group consisting of esters, nitriles, ketones and their mixtures and, on the other hand, a nonsolvent for CL20 consisting of at least one nonflammable HFE, said solvent for CL20 being more volatile than said nonsolvent (said solvent and said nonsolvent being obviously, in the proportions where they are involved, miscible for making up said mixtures, for making up said saturated solution),

the seeding of this saturated solution with a few crystals of hexanitrohexaazaisowurtzitane, then

the concentrating of the seeded solution by complete or partial evaporation of the solvent.

Advantageously, the seeding of said saturated solution is carried out with hexanitrohexaazaisowurtzitane crystals of c polymorphic form in order to obtain, at the end of the process, a suspension of hexanitrohexaazaisowurtzitane crystals of ε polymorphic form in a liquid phase. According to this alternative embodiment, said solution is preferably saturated with CL20 of another polymorphic form than the epsilon form.

Said process is thus characterized in that said nonsolvent (in association with a solvent with which it is miscible) consists of at least one nonflammable HFE, advantageously of at least one segregated nonflammable HFE, very advantageously of 2-trifluoromethyl-3-ethoxydodecafluorohexane.

The contents of solvent (less than 15% by weight) and crystals (in suspension) of the suspension obtained very obviously depend on the exact embodiment of the concentrating stage (on the desired degree of evaporation of the solvent). If it is desired to obtain suspensions which are virtually devoid, indeed even devoid, of solvent, the process of the invention advantageously comprises, in addition (on conclusion of the implementation of the concentrating stage), the washing, with said at least one nonflammable HFE, of the suspension obtained on conclusion of the concentrating of the seeded solution.

Advantageously, the solvent used in the process of the invention is chosen from ethyl acetate and acetone. Very advantageously, said solvent is ethyl acetate.

The suspensions of the invention are furthermore perfectly suitable as ingredients (sources of the charge of CL20) in processes for the manufacture of pyrotechnic objects. This is because they lend themselves to direct use in such processes for the manufacture of pyrotechnic objects. In contrast to water, normally used as phlegmatizing nonsolvent, said nonflammable HFEs can be easily extracted by evaporation during the implementation of said processes for the manufacture of pyrotechnic objects. Their latent heat of evaporation (88 kJ/kg for 2-trifluoromethyl-3-ethoxydodecafluorohexane) is much lower than that of water (2250 kJ/kg) and makes it possible to extract them by distillation during the processes for the manufacture of the pyrotechnic objects (in contrast, the extraction of the water necessarily has to be carried out by drying before carrying out the manufacturing process). After extraction, said at least one nonflammable HFE, which remains in residual amounts, is inert and does not interact with the ingredients of the pyrotechnic objects (in contrast to water, capable of reacting even in a very small amount with said ingredients).

According to its third subject matter, the present invention thus relates to the manufacture of pyrotechnic objects including hexanitrohexaazaisowurtzitane crystals. The process for the manufacture, according to the invention, of said objects comprises, conventionally, the introduction and the kneading of the ingredients (binder, crosslinking agent, charge(s), and the like) in a kneader (for forming a paste). Characteristically, in the context of said process, the CL20 crystals are not introduced after having been dried but in a suspension according to the first subject matter of the present invention. Said suspension is used as source of said crystals, as ingredient or starting material, and the like. Thus, characteristically, the process for the manufacture of pyrotechnic objects of the invention comprises:

the introduction of a suspension of the invention into the kneader, then, downstream,

the complete or virtually complete extraction under vacuum of the liquid phase of said suspension.

The CL20 crystals are introduced into the kneader in a suspension (of the invention), i.e. wetted (by said at least one nonflammable HFE), and phlegmatized (by said at least one nonflammable HFE), and the liquid phase of said suspension (predominantly composed, indeed essentially composed, indeed even exclusively composed, of said at least one nonflammable HFE) is subsequently evacuated by extraction, extraction under vacuum, carried out on the kneaded paste.

Advantageously, in order to facilitate the implementation of the stage of extraction under vacuum of said liquid phase, preference is given, among nonflammable HFEs, to those exhibiting the lowest saturated vapor pressure.

The great advantage of the suspensions of the invention is thus that they can be stored, transported and used in the manufacture of pyrotechnic objects as is (with the nonsolvent (indeed with said nonsolvent and small amounts of solvent) used during their preparation (by a solvent/nonsolvent process)).

It has been understood that the suspensions of the invention are conveniently used in the manufacture of pyrotechnic objects and that generally, before such a use (before they are introduced into the kneader), they are stored or transported or, successively, stored and then transported or, successively, transported and then stored.

The invention is now illustrated, in a way in no way limiting, by the appended figures and following examples (solubility study, implementation of the process of the invention, characterization of a suspension of the invention showing the phlegmatizing effect of 2-trifluoromethyl-3-ethoxydodecafluorohexane and implementation of an extraction of the novel nonsolvent of the invention as a mixture with a binder (extraction such as that which can be carried out in a process for the manufacture of pyrotechnic objects)).

The 2-trifluoromethyl-3-ethoxydodecafluorohexane used is HFE 7500, sold by 3 M.

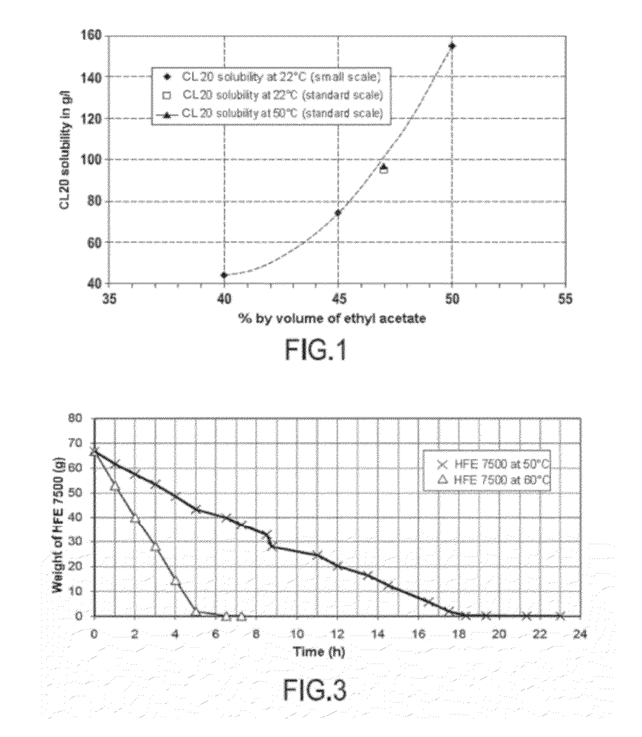

FIG. 1 shows the solubility of CL20 in an ethyl acetate/HFE 7500 mixture.

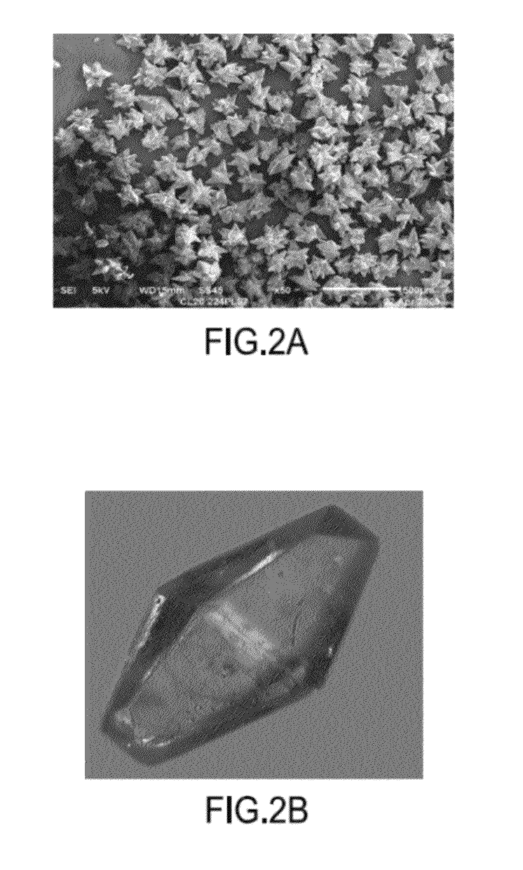

FIG. 2A shows a scanning electron microscopy image and FIG. 2B shows an optical microscope image (at higher magnification) of crystals of CL20ε obtained by the process of the invention (see point B below).

FIG. 3 shows the curves for evaporation, in a rotary evaporator, of HFE 7500 as a mixture with a binder (see point D below).

### POINT A

### Preliminary Study of the Solubility of Hexanitrohexaazaisowurtzitane in HFE 7500 and in an Ethyl Acetate/HFE 7500 Mixture

Ethyl acetate and HFE 7500 are completely miscible.

The solubility of CL20 in the nonsolvent HFE 7500 and in a solution composed of a mixture of ethyl acetate (solvent for CL20) and HFE 7500 (nonsolvent for CL20) is studied.
The solubility of CL20 in the nonsolvent HFE 7500 was measured, at 20° C., by liquid chromatography (HPLC), at 0.012 g/100 g of HFE 7500.
With reference to the processes for the crystallization of CL20 of the prior art (processes developed on the industrial scale), it is advantageous to target a solubility of approximately 100 g of CL20 dissolved per liter of the solution consisting of a mixture of ethyl acetate (solvent for CL20) and HFE 7500 (nonsolvent for CL20).

Tests carried out on a small scale (volume of solution less than 100 ml), at 20° C., presented in table 1, first of all made it possible to basically define an appropriate mixture ratio interval.

TABLE 1

| % by volume of ethyl | % by volume of | Solubility of CL20 in g/l |
|---|---|---|
| acetate | HFE 7500 | (20° C.) |
| 40 | 60 | 44 |
| 45 | 55 | 75 |
| 50 | 50 | 155 |

A solubility of approximately 100 g per liter of CL20 is obtained for a mixture comprising from 45 to 50% by volume of ethyl acetate.

The choice was thus directed towards a 47/53% by volume ethyl acetate/HFE 7500 mixture for a more thorough solubility study in a thermostatically-controlled reactor with a capacity of 5 l. The solubilization was monitored, step by step, using a Lasentec® probe in situ.

Thus, 1.5 l of 47/53% by volume ethyl acetate/HFE 7500 solution (i.e. respectively 634.5 g of ethyl acetate and 1293.4 g of HFE 7500) are prepared and are thermostatically controlled at 20° C. The CL20 is then added in small amounts and its dissolution is monitored via the measurements of the in situ probe. The amount of CL20 which can be dissolved in the mixture is 143 g, which thus represents a solubility of 95.3 g per liter of unsaturated solution.

The mixture is heated to 50° C. and the preceding procedure is repeated for the evaluation of the solubility. The solubility is measured at 96.6 g/l of solution. It is thus virtually constant with the temperature for the given 47/53% by volume ethyl acetate/HFE 7500 mixture. The various measurements carried out are given on the graph of FIG. 1.

### POINT B

### Implementation of the Process of the Invention (Example)

Following the teaching of point A above, crystallization by evaporation is carried out in a reactor with a volume of 2 l starting from a 47/53% by volume ethyl acetate/HFE 7500 medium comprising 470 ml of ethyl acetate and 530 ml of HFE 7500 saturated with CL20 (i.e. with 95.3 g of dissolved CL20).

There is found, as specific component (with regard to a conventional distillation setup), the stirrer (Lightnin A-310 propeller mixer) which has a thin profile, which does not cause significant breaking, with, however, a good pumping capacity, making it possible to overcome the separation by settling of the crystals under rotary conditions while avoiding breakage at the blade end. The diameter of the agitator with respect to the vessel diameter is 64 mm/100 mm. Its positioning in the reactor (distance between the agitator and the vessel bottom) was also defined by rules of chemical engineering. The degree of filling of the reactor does not exceed half. All this makes it possible, during the operation, to continually cause the crystals to pass into the growing phase at the evaporation interface (place where the solution becomes desaturated) without excessively stressing them “mechanically”.

After introduction, into the reactor, of the starting solution saturated with CL20, the bulk temperature is brought to 60° C. and then the seed (10 g of CL20ε with a mean size of 30 μm) is introduced. The stirring speed is 480 rev/min (which corresponds to a power per unit of volume, P/V=0.21 W/l, to a peripheral speed of the stirrer, PS=1.6 m/s, and to a pumping rate, PR=1.2 l/s). The ethyl acetate is then gradually distilled off over 1 h 45 by adjusting an appropriate reflux ratio and an appropriate vacuum gradient from 700 to 490 mbar, a function of the composition in the reboiler. After the ethyl acetate has been completely removed from the reboiler, the pressure is brought back to atmospheric pressure and the suspension of CL20ε in HFE 7500 is discharged.

In order to analyze, by electron microscopy, the CL20ε crystals of the suspension, the suspension is filtered and the retentate is pulled dry. The product is subsequently dried in an oven.

A crystalline product, with an “off white” color, composed of CL20ε crystals with a mean size of 100 μm, as shown in FIGS. 2A and 28, is recovered on the filter.

### POINT C

### Phlegmatization of CL20ε in Suspension in HFE 7500

The characterizations of the phlegmatization of the CL20ε by HFE 7500, which phlegmatization is studied on HFE 7500/CL20ε mixtures, with CL20ε having a particle size of 35±15 μm, at different contents by weight, are presented in table 2 below. The phlegmatization was evaluated by an impact sensitivity *(ISI) test, a friction sensitivity **(FSI) test, a sensitivity to electric spark ignition ***(ES) test and a deflagration to detonation transition ****(DDT) test. *Impact sensitivity (ISI) test: the test carried out corresponds to that described in the standard NF T 70-500, itself similar to the UNO test 3a)ii) resulting from the “Recommendations on the Transport of Dangerous Goods—Manual of Tests and Criteria, Fourth revised edition, ST/SG/AC.10/11/Rev.4, ISBN 92-1-239083-8ISSN 1014-7179”. The energy producing 50% (Bruceton method of treatment of the results) of positive results for an explosive material subjected to the impacts of a weight is determined by a minimum series of 30 trials. The test material is confined in a steel device composed of two rollers and a guide ring. By modifying the mass and the drop height of the weight, it is possible to vary the energy from 1 to 50 J. Due to the small amount of material available for some of the products tested, only a reduced number of reproducibility tests were carried out for said products, in comparison with the recommendations of the standard NF T 70-500.**Friction sensitivity (FSI) test: the test carried out corresponds to that described in the standard NF T 70-503, itself similar to the UNO 3b)ii) test. The force producing 50% of positive results for an explosive material subjected to friction is determined by a minimum series of 30 trials using the Bruceton method. The test material is placed on a porcelain plate of defined roughness, driven with only a to-and-fro movement with an amplitude of 10 mm at the rate of 7 cm/s offload, with respect to a porcelain peg resting on the material. The force applied to the porcelain peg which is supported on the material can vary from 7.8 to 353 N. Due to the small amount of material available for some of the products tested, only a reduced number of reproducibility tests were carried out for said products, with respect to the recommendations of the standard NF T 70-500.***Sensitivity to electric spark ignition (ES) test: the test carried out is a test developed by the Applicant Company without NF or UNO equivalent. The test material, placed in a dish with a diameter of 10 mm and a height of 1.5 mm, is positioned between two electrodes and is subjected to an electric spark of variable energy from 5 to 726 mJ. It is observed whether or not a pyrotechnic event has occurred and the energy threshold at which the material no longer ignites is determined. This value is confirmed by 20 successive trials. Due to the small amount of material available for some of the products tested, only a reduced number of reproducibility tests were carried out for said products.****Deflagration to detonation transition (DDT) test: this test is used to determine the transition possibilities of an explosive contained in a semi-closed steel tube (with an internal diameter of 40 mm, a thickness of 4 mm and a length of 200 and/or 400 mm. The lower part of the tube is closed by welding with a plate of the same nature with dimensions of 150×150×4 mm³) following local surface ignition (the ignition device is composed of a nickel/chromium (80/20) resistance wire with a diameter of 0.4 mm and a length of 5 to 10 mm).

TABLE 2

| | | CL20 ε/ | CL20 ε/ | CL20 ε/ |
|---|---|---|---|---|
| | CL20 ε | HFE 7500 20% | HFE 7500 35% | HFE 7500 50% |
| | dry | by weight | by weight | by weight |
| ISI | 1.9 | 1.4 | 7.5 | (7+/30) |
| (J) | | | | to 50.1 |
| FSI | 80 | 89 | 83 | 114 |
| (N) | | | | |
| ES | from 413 | >726 | >726 | >726 |
| (mJ) | to 726 | | | |
| DDT | 50 | 150 | >350 | >350 |
| mm) | (combustion) | (combustion) | (combustion) | (combustion) |
| | 75 | 175 | | |
| | (deflagration) | (deflagration) | | |
| | 100 | | | |
| | (detonation) | | | |

The result of the trial is assessed from the appearance of the steel tube. Three cases may be distinguished:

Combustion: Intact or swollen tube (negative trial).

Deflagration: Tube fragmented into large splinters (positive trial).

Detonation: Fragmented tube with the presence of small splinters (positive trial).

The following are recorded:

the greatest height (expressed in mm) at which two negative trials were obtained, it being known that a positive result was obtained at the higher step (+25 mm).

the lowest height (expressed in mm) at which a positive trial was obtained.

The phlegmatizing effect is not demonstrated for 20% of HFE 7500 by weight incorporated in CL20. One of the probable causes originates from the difficulty in obtaining a homogeneous mixture for such a low level of wetting starting from a dry product. The values obtained in the different tests are similar to those obtained with regard to dry CL20.

On the other hand, starting from 35% by weight of HFE 7500, the phlegmatizing effect is more pronounced. The ISI is 7.5 J (against 1.9 J with regard to the same CL20 dry) and there is combustion for the high value of the DDT test. Nevertheless, a few difficulties remain in preparing a homogeneous mixture.

Starting from 50%, the phlegmatizing effect is proven. The high values of the different tests carried out are reached and a phlegmatizing effect is also recorded with regard to the sensitivity to attacks of friction type (114 N versus 80-90 N for CL20 dry).

### POINT D

### Extraction of HFE 7500 as a Mixture with a Binder, Capable of Being Employed in a Process for the Manufacture of Pyrotechnic Objects

The possibility of extracting HFE 7500 as a mixture with a binder of poly(diethylene glycol adipate) (PDEGA) type was evaluated in a laboratory rotary evaporator. The extraction of HFE 7500 in a process for the manufacture of pyrotechnic objects is thus simulated.

The tests for the extraction of HFE 7500 were carried out on a mixture comprising, by weight, ⅓ of PDEGA and ⅔ of HFE 7500.

A weight of 100 grams of mixture was placed in the rotary evaporator. The operating conditions are given below:

pressure: 100® 130 mbar

temperature: 50 and 60° C.

extraction time: until complete disappearance of the HFE 7500 by measurement of loss in weight

recording the weight evaporated every hour.

in order to monitor the amount of HFE 7500 extracted over time, the round-bottomed flasks are weighed every hour. The change in the loss in weight of the mixture was thus monitored until virtually all of the assumed amount of HFE 7500 had disappeared.

The graph in FIG. 3 shows the loss in weight (of the PDEGA/HFE 7500 mixtures) related to the evaporation of HFE 7500 for the two test temperatures (for an equivalent vacuum): the rate of extraction is 3.5 times faster at 60° C. than at 50° C. and the residual content of HFE 7500 after extraction is less than 0.2% of that of the initial HFE 7500.

In order to confirm with greater accuracy the content of HFE 7500 present at the end of extraction, the mixture recovered at the end of the test carried out at 50° C. was analyzed by gas chromatography. The residual HFE 7500 content measured by this method is 0.02%.

The possibility of extracting HFE 7500 from a propellant paste during kneading, using a vacuum kneading device equipped with a condenser, has also been demonstrated.

1. A suspension of hexanitrohexaazaisowurtzitane crystals in a liquid phase, wherein said liquid phase is composed, to at least 85% by weight, of a nonsolvent for hexanitrohexaazaisowurtzitane, said nonsolvent consisting of at least one nonflammable hydrofluoroether, and includes from 0 to less than 15% by weight of an organic solvent for hexanitrohexaazaisowurtzitane, which is more volatile than said nonsolvent, chosen from the group consisting of esters, nitriles, ketones and their mixtures.

2. The suspension as claimed in claim 1, wherein said solvent, present at less than 15% by weight in said liquid phase, is chosen from ethyl acetate and acetone.

3. The suspension as claimed in claim 1, wherein said nonflammable hydrofluoroether is chosen from segregated nonflammable hydrofluoroethers.

4. The suspension as claimed in claim 1, wherein said nonflammable hydrofluoroether consists of 2-trifluoromethyl-3-ethoxydodecafluorohexane.

5. The suspension as claimed in claim 1, wherein it includes at least 35% by weight, of said at least one nonflammable hydrofluoroether.

6. A process for producing a suspension of hexanitrohexaazaisowurtzitane crystals as claimed in claim 1, comprising:
preparation of a saturated solution of hexanitrohexaazaisowurtzitane of any polymorphic form in a mixture comprising an organic solvent for hexanitrohexaazaisowurtzitane chosen from the group consisting of esters, nitriles, ketones and their mixtures and a nonsolvent for said hexanitrohexaazaisowurtzitane consisting of at least one nonflammable hydrofluoroether, said solvent being more volatile than said nonsolvent,
seeding said saturated solution with a few crystals of hexanitrohexaazaisowurtzitane, then
concentrating said seeded solution by complete or partial evaporation of the solvent.

7. The process as claimed in claim 6, further comprising washing, with said at least one nonflammable hydrofluoroether, of the suspension obtained on conclusion of said concentrating of said seeded solution.

8. The process as claimed in claim 6, wherein said solvent is chosen from ethyl acetate and acetone.

9. A process for the manufacture of pyrotechnic objects including hexanitrohexaazaisowurtzitane crystals, comprising:
introduction of a suspension as claimed in claim 1 into a kneader, and
complete or virtually complete extraction under vacuum of the liquid phase of said suspension.

10. The process as claimed in claim 9, further comprising:
storage or transportation, or storage and transportation, or transportation and storage of said suspension, before it is introduced into said kneader.

11. The suspension as claimed in claim 1, wherein said solvent, present at less than 15% by weight in said liquid phase, consists of ethyl acetate.

12. The suspension as claimed in claim 1, wherein said solvent consists of ethyl acetate and said nonsolvent consists of 2-trifluoromethyl-3-ethoxydodecafluorohexane.

13. The suspension as claimed in claim 1, which includes at least 50% by weight of said at least one nonflammable hydrofluoroether.

14. The process as claimed in claim 6, wherein said solvent consists of ethyl acetate.
